(19) United States
(12) Patent Application Publication
(10) Pub. No.: US 2025/0361406 A1
(43) Pub. Date: Nov. 27, 2025

(54) RADIATION-CURING COMPOSITION FOR THE PRODUCTION OF DENTAL COMPONENTS

(71) Applicant: KULZER GMBH, Hanau (DE)

(72) Inventors: Dennis BARTHER, Seligenstadt (DE); Christoph MEIER, Bruchköbel (DE)

(21) Appl. No.: 19/214,614

(22) Filed: May 21, 2025

(30) Foreign Application Priority Data
May 21, 2024 (EP) 24176989.2

(51) Int. Cl. C09D 4/06; A61C 13/00; B33Y 10/00; B33Y 70/00; B33Y 80/00; C08F 283/01; C09D 151/08

(52) U.S. Cl. CPC C09D 4/06; A61C 13/0004; A61C 13/0019; B33Y 10/00; B33Y 70/00; B33Y 80/00; C08F 283/01; C09D 151/08

(57) ABSTRACT

The invention relates to a radiation-curing composition for the manufacture of dental components using the DLP process or SLA process, comprising, based on the total mass of the radiation-curing composition (i) one or more radically polymerizable monomers in a combined mass fraction of 60% or more, (ii) one or more hexaarylbiimidazole compounds in a combined mass fraction in the range of 0.1 to 5%, and (iii) one or more mercaptotetrazole compounds in a combined mass fraction in the range of 0.1 to 5%, wherein the combined mass fraction of fillers in the radiation-curing composition is less than 30%.

The invention relates to a radiation-curing composition for the production of dental components using the DLP process or SLA printing processes, a dental component producible by radiation-induced polymerization of a radiation-curing composition, and a use of a specific initiator system in a radiation-curing composition for improving the mechanical properties of the dental components which can be produced therefrom using the DLP process or SLA process.

The technological progress and ongoing digitalization that have changed the industrial landscape over the last decade have also influenced the field of dental technology, fundamentally changing the everyday work of dentists and dental technicians. For decades, many types of dental prostheses, such as dental crowns, bridges, partial and full dentures or inlays, as well as orthodontic treatment appliances, such as splints, were mainly produced manually by dental technicians.

These days, the traditional process is increasingly being supported or replaced by the use of computer-aided production and manufacturing processes. This technology is sometimes also referred to by specialists as digital prosthetics.

In most cases, CAD/CAM processes are an integral part of computer-aided production. The term CAD refers to computer-aided design, which in a broader sense describes the creation of a digital component that can be modified directly on the computer by the user if necessary. The term CAM refers to computer-aided manufacturing, i.e. the conversion of a CAD-generated component into a code that can be used, for example, to control a metal-cutting machine or a 3D printer. In the field of dental technology, the use of intraoral scanners, which can transmit a precise image of the patient's oral cavity to the computer without requiring contact, has become an increasingly established method for the creation of CAD components.

Additive manufacturing processes, in which products are manufactured from shapeless materials without the use of special tools and based on CAD-generated computer data sets, are particularly important for dental technology. These 3D printing processes, which are sometimes also referred to as “rapid prototyping” processes, replace or supplement many work steps in the manufacture of dental components in the field of dental technology today.

Stereolithography (SLA) and digital light processing (DLP) play a prominent role in dental 3D printing processes. In these processes, a radiation-curing composition is applied and cured layer by layer at the desired points using spatially resolved, targeted radiation. In this process, the resulting component is lowered gradually into the composition or raised gradually out of it, for example, so that after each increment only a thin film of the radiation-curing composition is applied on top of the last layer, which corresponds approximately to the thickness of the next layer to be polymerized. The basic principle of the SLA and DLP processes are similar, but are quite different in terms of the equipment used. For example, the SLA process uses a laser that scans over the structure to be produced one layer at a time, whereas the DLP process uses a suitable projection technique to simultaneously expose an entire surface. The principle of the SLA and DLP processes is known from the state of the art and is described, for example, in U.S. Pat. No. 4,575,330 A or WO 2014078537 A1. The use of additive manufacturing processes in dental technology is known, for example, from the documents U.S. Pat. No. 979,554 B2, WO 2023126943 A2, DE 102016107935 A1 and DE 1020122011371 A1 and is also described in EP 3020361 B1.

Despite the increasing use of corresponding radiation-curing compositions, there is continued interest in the field in optimizing the materials used. In particular, the aim is to optimize the processing properties of the radiation-curing compositions and at the same time to improve the properties of the materials that can be produced from them by curing, especially their mechanical properties.

When designing radiation-curing compositions for use in 3D printing processes, the initiator system often plays a decisive role. The initiators used must not only ensure that the polymer has good mechanical properties but also have advantageous polymerization kinetics. In addition to the polymerization speed, which is dependent on the initiators used, the question of polymerization depth is of particular importance. It is also important to know what initial dose of radiation is required to initiate the polymerization reaction and what average layer thicknesses can be achieved with a given radiation dose. In particular, the depth of polymerization correlates in part with the wavelength of the electromagnetic radiation used for initiation. However, this wavelength also has other important implications for the 3D printing devices used. The efficiency of a radiation-curing composition in 3D printing depends largely on how well the 3D printing equipment used can provide the initiator system's optimum wavelengths.

A number of advantageous initiator systems have been identified in the past in light of the conflicting requirements described above.

Phosphine oxide-based Norrish type I initiators, such as phenylbis(2,4,6-trimethylbenzoyl) phosphine oxide (BAPO), diphenyl(2,4,6-trimethylbenzoyl) phosphine oxide (TPO) and ethyl(2,4,6-trimethylbenzoyl) phenylphosphinate (TPO-L), are widely used due to their broad absorption spectrum. Compared to Norrish type II initiators, such as the equally established camphorquinone/amine system, however, the maximum of the absorption spectrum is mostly localized in the shorter-wave UV range and therefore only enables low curing depths and limited usability on some 3D printers. A high curing depth is particularly advantageous for post-curing and produces better mechanical properties of the printed materials.

Another disadvantage of using Norrish type I initiators is that the polymerization reactivity of the material being printed can only be poorly controlled, as it is a one-component initiator system.

The choice of suitable initiators is becoming increasingly difficult as awareness of environmental and/or health aspects grows, because many of the known state-of-the-art initiator systems are now viewed critically from an environmental and/or health perspectives and their use is already restricted in some countries or is not unlikely to be restricted in the near future. Despite their good performance in terms of mechanical properties and reactivity and their versatile use, phosphine oxide-based initiators are suspected of having toxic reproductive effects on the human body. TPO, for example, has already been included in the European Chemicals Agency's (ECHA) list of “Substances of very high concern” and classified as potentially carcinogenic, mutagenic and toxic for reproduction (CMR) (class 2), which calls into question the general use of this class of substances in medical devices.

The primary task of this invention is to eliminate or at least mitigate the disadvantages of the prior art.

In particular, an objective of this invention is to provide a radiation-curing composition whose initiator system is less problematic from an environmental and/or health perspective than the solutions known from the prior art, it being a desirable requirement that the initiator system should preferably not be subject to any regulatory restrictions worldwide, in particular relating to the additive manufacturing of medical devices.

In this respect, a key objective of this invention is that the radiation-curing composition disclosed should have advantageous polymerization kinetics, in particular with regard to the radiation dose required to initiate the process and with regard to the average layer thickness that can be achieved at a given radiation dose.

Another objective of this invention is that the radiation-curing composition to be disclosed should be able to be converted into advantageous polymers which have advantageous mechanical properties which also qualify them for high-performance applications in the dental field.

In this respect, it was an objective of this invention that the radiation-curing composition to be disclosed should, as far as possible, be comparable in terms of the polymerization kinetics and the mechanical properties of the polymer with radiation-curing compositions known from the prior art which use established initiator systems, in particular those of Norrisch type I, to the extent that these properties are at least at a comparable level, it being desirable for these properties to even be improved, at least in part, so that the conflicts of objectives in this respect at present are better resolved.

It was a desirable requirement of this invention that the radiation-curing compositions to be disclosed should in particular also be able to be processed efficiently in modern 3D printers, i.e. that curing at wavelengths of 385 nm or less should be efficiently possible, it being a desirable feature that the initiator systems to be disclosed should have a sufficiently wide excitation range that they can be cured with a wide range of existing 3D printers, in particular also those operating at wavelengths of about 405 nm.

An additional objective of this invention to produce a dental component which can be produced from the radiation-curing compositions to be described and which can not only be produced in a particularly time-and cost-efficient manner, but which also has excellent mechanical properties and is also considered advantageous in terms of environmental and health considerations.

It is a further objective of this invention to disclose the use of a specific initiator system for improving the mechanical properties of dental components produced by 3D printing.

The inventors of this invention found that the objectives described above can, surprisingly, be solved if an initiator system which uses hexaarylbiimidazole compounds in conjunction with mercaptotetrazole compounds, as defined in the claims, is used in a radiation-curing composition which has a high proportion of radically polymerizable monomers and a comparatively low proportion of filler materials.

Unexpectedly, the use of a corresponding initiator system in the specific low-filler, radiation-curing compositions was found to have advantageous curing kinetics, particularly with regard to the initial radiation dose required, and the polymers obtained were also found to have advantageous mechanical properties. In combination with the other components of the radiation-curing composition, the use of the initiator system as a whole provides performance properties that are at least comparable to those of established initiator systems known from the prior art and in some cases even improved. The combination of hexaarylbiimidazole compounds with mercaptotetrazole compounds is preferable from an environmental and health point of view when compared to many of the initiator systems known from the state of the art, so that the identified initiator system is particularly advantageous for use in the additive manufacturing of medical devices.

The above tasks are therefore solved by the object of the invention as defined in the claims. The preferred embodiments according to the invention are shown in the subclaims and the following explanations.

These embodiments, which are designated as preferred below, are combined with features of other embodiments designated as preferred into particularly preferred embodiments. Combinations of two or more of the embodiments described below as particularly preferred are therefore particularly preferred. Embodiments in which a feature of one embodiment that is described as preferred to any extent is combined with one or more further features of other embodiments that are described as preferred to any extent are particularly preferred. Features of preferred dental components and uses are derived from the features of preferred radiation-curing compositions.

Insofar as both specific amounts or proportions of this element and preferred embodiments of the said element are disclosed below for an element, for example for the radically polymerizable monomers or the fillers, the specific amounts or proportions of the preferred embodiments of the elements are also disclosed in particular. In addition, it is disclosed that in the corresponding specific total quantities or total proportions of the elements, at least some of the elements may be configured in a preferred manner and, in particular, that elements configured in a preferred manner within the specific total quantities or total proportions may in turn be present in the specific quantities or proportions.

Particularly preferred embodiments of the invention are described in the example embodiments. In view of this, particularly preferred embodiments of the invention have two or more, preferably three or more, most preferably four or more, of the preferred features of the invention disclosed below, which are also implemented in the embodiment examples.

The invention relates in particular to a radiation-curing composition for the production of dental components using the DLP process or SLA process, comprising, based on the total mass of the radiation-curing composition:
(i) one or more radically polymerizable monomers in a combined mass fraction of 60% or more,
ii) one or more hexaarylbiimidazole compounds in a combined mass fraction in the range from 0.1 to 5%, and
iii) one or more mercaptotetrazole compounds in a combined mass fraction in the range from 0.1 to 5%,
wherein the combined mass fraction of fillers in the radiation-curing composition is less than 30%.

The radiation-curing composition according to the invention is suitable and intended for the production of dental components in a 3D printing process, namely a DLP process or SLA process. Radiation-curing compositions for this purpose are known, as are the devices that can be used for the DLP process or SLA process, and corresponding products and devices are commercially available from various suppliers, for example from the company Kulzer.

The terms “DLP method” and “SLA method” refer to the methods of digital light processing and stereolithography described above, which are methods known to a specialist. The suitability specification of the radiation-curing composition for the manufacture of dental components in these processes consist of in particular a functional requirement regarding the viscosity of the material, insofar that this should be sufficiently low. This excludes many radiation-curing compositions that are used elsewhere in the dental field, especially those with high filler content, such as ceramic slurries. With respect to the liquid properties of a radiation-curing composition according to the invention, it is preferred that the radiation-curing composition has a dynamic viscosity at 23° C. in the range from 0.001 to 10 Pa s, preferably in the range from 0.5 to 5 Pa s, particularly preferably in the range from 0.7 to 2.5 Pa s. The viscosity of the non-cured radiation-curing compositions is determined in the context of this invention using an Anton Paar-Physica MCR301 rheometer at 23° C. (interchangeable plate I-PP-50/SS smooth and measuring cone CP25-1, 25 mm, 1°). The width of the opening is given by the angle and diameter of the cone. The measurement is carried out under constant rotation and a shear rate of 100 1/s. A total of 10 measuring points are recorded. The duration of a measuring point is 6 s with a section time of 60 s.

However, the above suitability does not exclude the possibility that the radiation-curing compositions according to the invention could also be used in other additive manufacturing processes, for example in the context of “ink jet” technology. The inventors believe, however, that the radiation-curing compositions according to the invention will be used in future practice primarily with the DLP process. Accordingly, according to the invention it is preferred to use a radiation-curing composition suitable for the production of dental components using the DLP process.

In the context of this invention, the defined components of the radiation-curing composition are each used as “one or more” in accordance with the understanding of those skilled in the art. The term “one or more” refers to the chemical nature of the corresponding compounds and not to the quantity of the substance, as is customary in the industry.

Insofar as mass fractions are stated in the context of the invention, these are stated in the customary manner as the combined mass fractions of the one or more components, with the mass fractions of the correspondingly formed components taken together fulfills the corresponding criteria.

Unless otherwise defined in individual cases, the mass fractions defined for the components of the radiation-curing composition in the context of this invention refer in each case to the total mass of the radiation-curing composition. A specialist understands that the mass fractions are defined with the stipulation that the total mass fraction of the radiation-curing composition add up to 100%, so that defined upper limits of a component are to be adjusted if necessary, so that together with the lower limits of the other obligatory components they add up to 100%.

For the purposes of this invention, the term dental components refers in principle to all components and three-dimensional structures which are produced in the field of dental technology as an aid, intermediate stage or end product, i.e. irrespective of the underlying dental indication. However, due to the advantageous mechanical properties of the dental components which can be produced by curing the radiation-curing compositions, this invention is particularly suitable for the production of dental components for permanent dental applications, i.e. in the patient's mouth. An example is therefore a radiation-curing composition according to the invention, wherein the dental components are selected from the group consisting of dental prostheses, dental models, gingival masks, occlusal splints, CAD-to-cast molds, impression trays, drilling templates, temporary and permanent crowns, aligners, bridges, onlays, inlays and veneers. However, a radiation-curing composition according to the invention is preferred wherein the dental components are selected from the group consisting of dental prostheses, temporary and permanent crowns, aligners, bridges, onlays, inlays and veneers.

The term “radiation-curing”, which is used above to characterize the composition, corresponds to the technical term commonly used in the industry, although terms such as “radiation-curable”, “radiation-crosslinking” or similar expressions are also used in an equivalent manner. The term is to be understood as the property of the composition to cure through the application of electromagnetic radiation, in particular in the ultraviolet or visible light range, in that the radiation induces the polymerization of radically polymerizable monomers in the composition using an initiator. The wavelength of the radiation used for this is usually in the visible light range or the adjacent wavelength ranges in the UV range, with wavelengths in the blue and ultraviolet range used particularly frequently. In accordance with the understanding of a specialist, radiation curability is achieved by combining radically polymerizable monomers with initiators as defined above. In regard to the specific initiators it is preferred that a radiation-curing composition according to the invention is designed to cure upon irradiation of the radiation-curing composition with electromagnetic radiation in the wavelength range of 200 and 420 nm, preferably in the wavelength range of 250 to 415 nm, particularly preferably in the wavelength range of 300 to 410 nm. In other words, a radiation-curing composition according to the invention is preferred, wherein the photo-initiator system comprising the hexaarylbiimidazole compounds and the mercaptotetrazole compounds is designed to initiate polymerization of the polymerizable monomers when the radiation-curing composition is irradiated with electromagnetic radiation in the wavelength range from 200 to 420 nm, preferably in the wavelength range from 250 to 415 nm, particularly preferably in the wavelength range from 300 to 410 nm.

In addition to the initiators discussed in more detail below, the radically polymerizable monomers to be used according to the invention are an important component which determines the radiation curability. The radically polymerizable monomers are characterized by their ability to crosslink with each other in a chain reaction after initiation by a radical initiator, whereby the radical initiator in radiation-curing compositions is usually provided by the initiator(s). The basic principle of this invention is not limited to certain radically polymerizable monomers, but can be applied to all radically polymerizable monomers, most of which have a terminal unsaturated double bond through which the radical polymerization can take place. These monomers, which can be monofunctional or multifunctional, are generally selected by the specialist based on the physico-chemical and technical application properties required for the dental component to be produced. In the field of dental chemistry, (meth) acrylates in particular are extremely important as monomers, whereby the term (meth) acrylates refers to both acrylates and methacrylates in accordance with the understanding of the specialist. These monomers, which are frequently used in the field of dental chemistry, are known to a specialist and are disclosed, for example, in EP 3020361 B1 or DE 3941629 C1. In this context, an example is a radiation-curing composition according to the invention, wherein the one or more radically polymerizable monomers are selected from the group consisting of (meth)acrylic acid, (meth)acrylates, (meth)acrylamides and other vinyl compounds, preferably selected from the group consisting of (meth)acrylic acid, (meth)acrylates and (meth)acrylamides, particularly preferably selected from the group consisting of (meth)acrylates. Preferred is a radiation-curing composition according to the invention, wherein the one or more radically polymerizable monomers are selected from the group consisting of monofunctional (meth)acrylates and polyfunctional (meth)acrylates.

Examples of suitable monofunctional (meth)acrylates are, for example, selected from the group consisting of substituted and unsubstituted alkyl methacrylates and substituted and unsubstituted cycloalkyl(meth)acrylates, preferably selected from the group consisting of dicyclopentanylmethyl acrylate, tertiobutylcyclohexl(meth)acrylate, 2 (2-ethoxyethoxy)ethyl(meth)acrylate, tetrahydrofurfuryl(meth)acrylate, octyldecyl(meth)acrylate, isobornyl(meth)acrylate, isodecyl(meth)acrylate and alkyl(meth)acrylates with up to 12 carbon atoms in the alkyl radical.

Examples of suitable polyfunctional (meth)acrylates are, for example, selected from the group consisting of urethane dimethacrylate, BisGMA, triethylene glycol di(meth)acrylates (TEGD [M]A), tricyclodecane dimethanol di(meth)acrylate (TCDDMD [M]A), bisphenol A ethoxylate (with different chain lengths) (Bis-EMA), 1,6-hexanediol di(meth) acrylate, polyethylene glycol di(meth)acrylate, 3-methyl-1,5-pentanediol di(meth)acrylate, Dipropylene glycol di(meth)acrylate, ester di(meth)acrylate, trimethylolpropane tri(meth)acrylate) (TMPT [M]A), tris(2-hydroxyethyl)isocyanurate tri(meth)acrylate, Di-pentaerythritol penta(meth)acrylate), ethoxylated or propoxylated trimethylolpropane tr(meth)acrylate, 2-propenoic acid, 1, 1′-[((octahydro-4,7, methano-1H-indene-5,diyl)bis(methyleneiminocarbonyloxy-2,1-ethanediyl)] ester (TCD-Di-HEA;CAS 861437 Nov. 8), 2-propeonic acid, 1,1′[(octahydro-4,7-methano-1H-indene-5-diyl)bis(methyleneoxycarbonylamino-2,1-ethanediyl)] ester (TCD-Di-UEA; CAS 945656-78-0), decanediol-1,10-dimethacrylate and urethane dimethacrylate as well as other urethane (meth) acrylates and thiourethane (meth) acrylates as disclosed, for example, in WO 2022248546 A1.

According to the inventors, particularly efficient radiation-curing compositions can be obtained by using or combining mono-and difunctional (meth) acrylates. Accordingly, a radiation-curing composition according to the invention is preferred, wherein the radiation-curing composition comprises at least one radically polymerizable monomer selected from the group consisting of monofunctional (meth) acrylates, and/or wherein the radiation-curing composition comprises at least one radically polymerizable monomer selected from the group consisting of polyfunctional (meth) acrylates, preferably difunctional (meth) acrylates. In the inventors' experiments, particularly advantageous property profiles were obtained when the initiator system according to the invention was used in combination with both monofunctional and difunctional monomers in the monomer component.

With regard to the choice of monomers, a radiation-curing composition according to the invention particularly preferred, wherein the radiation-curing composition comprises at least one radically polymerizable monomer selected from the group consisting of ethoxylated 2-bisphenol-A-dimethacrylates, tricyclodecane methanol acrylates, urethane dimethacrylates and carboxy-functionalized polyester acrylates, tricyclodecane methanol acrylates, urethane dimethacrylates and carboxy-functionalized polyester acrylates, wherein the radiation-curing composition preferably comprises two or more, more preferably three or more, particularly preferably four or more, different of these radically polymerizable monomers.

With respect to the basic content of the monomer component, a radiation-curing composition according to the invention is preferred, wherein the radiation-curing composition comprises the one or more radically polymerizable monomers in a combined mass fraction of 65% or more, preferably 70% or more, more preferably 75% or more, most preferably 85% or more. In addition or alternatively, a radiation-curing composition according to the invention is preferred, wherein the radiation-curing composition comprises the one or more radically polymerizable monomers in a combined mass fraction in the range from 60 to 96%, preferably in the range from 70 to 93%, more preferably in the range from 80 to 90%.

An essential aspect of the radiation-curing composition according to the invention is that the combined mass fraction of fillers is selected to be relatively low, wherein the inventors consider it preferable to tendentially select even lower filler contents. It is conceivable and also preferred for some applications if the radiation-curing composition according to the invention is completely free of fillers. Therefore, a radiation-curing composition according to the invention is preferred, wherein the radiation-curing composition comprises a combined mass fraction of 25% or less of fillers, preferably 20% or less, more preferably 15% or less.

Even if, as explained above, it is conceivable to make the radiation-curing composition according to the invention without fillers, the inventors consider it particularly preferable that a filler is deliberately added to the radiation-curing composition in order to achieve advantageous mechanical properties. Accordingly, a radiation-curing composition according to the invention is preferred, wherein the radiation-curing composition additionally comprises:
iv) one or more fillers in a combined mass fraction in the range from 0.05 to 29%,

In their own tests, the inventors succeeded in specifying particularly suitable mass fractions for the filler used in this regard which can achieve advantageous polymerization kinetics with excellent mechanical properties, when used in combination with the initiator systems to be used according to the invention. In particular, a radiation-curing composition according to the invention is preferred, wherein the radiation-curing composition comprises the one or more fillers in a combined mass fraction of less than 25%, preferably less than 20%, more preferably less than 15%. In addition or alternatively, also preferred is a radiation-curing composition according to the invention is preferred, wherein the radiation-curing composition comprises the one or more fillers in a combined mass fraction in the range from 0.1 to 25%, preferably in the range from 0.5 to 22.5%, more preferably in the range from 1 to 20%, most preferably in the range from 2 to 17.5%, particularly preferably in the range from 5 to 15%.

In the opinion of the inventors, fillers which are frequently used in the dental field for comparable products, e.g. dental glasses, can in principle be used for the implementation of this invention. However, the inventors' experiments have shown that particularly advantageous properties can be achieved with fillers made of silicon dioxide and zirconium dioxide, including in particular the filler also known as “precipitated silica”. Consequently, a radiation-curing composition according to the invention is preferred, wherein the radiation-curing composition, wherein the one or more fillers are selected from the group consisting of oxidic fillers, preferably selected from the group consisting of silicium dioxide and zirconium dioxide, more preferably selected from the group consisting of amorphous silicium dioxide, in particular selected from the group consisting of precipitated amorphous silicium dioxide. A radiation-curing composition according to the invention is preferred for most embodiments, wherein the one or more fillers are selected from the group consisting of particulate fillers.

An essential aspect of this invention is the interaction of two specific classes of compounds in the initiator system of the radiation-curing composition according to the invention. For this purpose, hexaarylbiimidazole compounds are combined with mercaptotetrazole compounds, which together act as a photo-initiator or co-initiator and cause the radiation-curability of the radiation-curing compositions according to the invention.

In their own extensive experiments, the inventors succeeded in identifying preferred ranges for the contents of the components of the initiator system to be used according to the invention, which can achieve excellent results when used in the DLP or SLA process. In the course of these studies, it has been identified that it is particularly advantageous to use the two compounds in at least equal mass fractions, although it is preferable to add more of the mercaptotetrazole compounds. Particularly preferred is a radiation-curing composition according to the invention, wherein the radiation-curing composition comprises the one or more hexaarylbiimidazole compounds in a combined mass fraction in the range from 0.15 to 4.5%, preferably in the range from 0.2 to 4.0%, more preferably in the range from 0.25 to 3.5%, most preferably in the range from 0.3 to 3.0%. In addition or alternatively, a radiation-curing composition according to the invention is also preferred, wherein the radiation-curing composition comprises the one or more mercaptotetrazole compounds in a combined mass fraction in the range from 0.15 to 4.5%, preferably in the range from 0.2 to 4.0%, more preferably in the range from 0.25 to 3.5%, most preferably in the range from 0.3 to 3.0%. In addition or alternatively, a radiation-curing composition according to the invention is preferred, wherein the quotient of the combined molar fraction of the one or more hexaarylbiimidazole compounds divided by the combined molar fraction of the one or more mercaptotetrazole compounds is 1.0 or less, preferably 0.8 or less, more preferably 0.6 or less, in particular 0.4 or less.

Hexaarylbiimidazole compounds and their use as photo-initiators are known from the prior art and are disclosed, for example, in US 2017/0266081 A1. The inventors' experiments have shown that it is possible to produce particularly efficient systems using substituted hexaarylbiimidazole compounds. Accordingly, a radiation-curing composition according to the invention is preferred, wherein the one or more hexaarylbiimidazole compounds are selected from the group consisting of hexaarylbiimidazole compounds having one or more, preferably two or more, more preferably three or more, most preferably four or more substituent-substituted aryl groups. Particularly preferred is radiation-curing composition according to the invention, wherein the substituents are selected from the group consisting of halogen atoms and alkoxy groups, preferably chlorine atoms and methoxy groups.

Particularly favorable results in the overall performance properties were achieved by the inventors in this respect and also through the combined use of two or more different hexaarylbiimidazole compounds, by which means the polymerization properties of the radiation-curing compositions according to the invention can be specifically adapted to the respective application requirements in particular. Therefore, a radiation-curing composition according to the invention is preferred, wherein the radiation-curing composition comprises two or more different hexaarylbiimidazole compounds.

Hexaarylbiimidazole compounds can in principle be designed differently with regard to the linkage of the imidazole rings. The inventors have achieved good results in particular with those compounds in which the linkage took place via an N—N bond or via a C—N bond, with the C—N linkage showing particularly advantageous results overall. For some applications a radiation-curing composition according to the invention is preferred, wherein the one or more hexaarylbiimidazole compounds are selected from the group consisting of hexaaryl-1,1′-biimidazole compounds, and/or wherein the one or more hexaarylbiimidazole compounds are selected from the group consisting of hexaarylbiimidazole compounds in which the imidazole rings are linked via a C—C bond. In most cases, however, a radiation-curing composition according to the invention is preferred, wherein the one or more hexaarylbiimidazole compounds are selected from the group consisting of hexaaryl-1,2′-biimidazole compounds, and/or wherein the one or more hexaarylbiimidazole compounds are selected from the group consisting of hexaarylbiimidazole compounds in which the imidazole rings are linked via a C—N bond.

In the course of the conducted experiments, the inventors were able to identify particularly suitable hexaarylbiimidazole compounds and also to identify three particularly powerful representatives of this compound class. First of all, a radiation-curing composition according to the invention is preferred, wherein the one or more hexaarylbiimidazole compounds selected from the group consisting of 2,2′-bis (2-chlorophenyl)-4,4′,5,5′-tetraphenyl-1,2′-biimidazole (o-CI-HABI), 2,2′-bis (2,4-dichlorophenyl)-4,4′,5,5′-tetraphenyl-1,2′-biimidazole (2,4-Cl-HABI), 2,2′-bis(3-chlorophenyl)-4,4′,5,5′-tetraphenyl-1,2′-biimidazole (3-Cl-HABI), 2,2′-bis (4-chlorophenyl)-4,4′,5,5′-tetraphenyl-1,2′-biimidazole (4-Cl-HABI), 2,2′-bis (phenyl)-4,4′-bi(2-chlorophenyl)-5,5′-biphenyl-1,2′-biimidazole, 2,2′-bis (phenyl)-4,4′-biphenyl-5,5′-bi(2-chlorophenyl)-1,2′-biimidazole, 2,2′-bis (phenyl)-4,4′,5,5′-tetra(2-chlorophenyl)-1,2′- biimidazole 2, 2′,4-tris(2-chlorophenyl)-5-(3,4-dimethoxyphenyl)-4′,5′-diphenyl-1, 2′-biimidazole and 2,2′,4-tris (2-chlorophenyl)-5-(3,4-dimethoxyphenyl)-4′,5′-diphenyl-1,1′-biimidazole. In addition or alternatively, a radiation-curing composition according to the invention is especially preferred, wherein the one or more hexaarylbiimidazole compounds are selected from the group consisting of 2,2′-bis(2-chlorophenyl)-4, 4′,5,5′-tetraphenyl-1,2′-biimidazole 2,2′,4-tris(2-chlorophenyl)-5-(3,4-dimethoxyphenyl)-4′,5′-diphenyl-1,2′-biimidazole and 2,2′,4-tris(2-chlorophenyl)-5-(3,4-dimethoxyphenyl)-4′,5′-diphenyl-1,1′-biimidazole. In addition or alternatively, a radiation-curing composition according to the invention is also especially preferred, wherein the radiation-curing composition comprises as hexaarylbiimidazole compounds 2, 2′-bis (2-chlorophenyl)-4,4′,5,5′-tetraphenyl-1,2′-biimidazole and/or 2,2′,4-tris (2-chlorophenyl)-5-(3, 4-dimethoxyphenyl)-4′,5′-diphenyl-1,2′-biimidazole and/or 2,2′,4-tris(2-chlorophenyl)-5-(3,4-dimethoxyphenyl)-4′,5′-diphenyl-1,1′-biimidazole, preferably 2, 2′-bis(2-chlorophenyl)-4,4′,5,5′-tetraphenyl-1,2′-biimidazole and 2,2′,4-tris(2-chlorophenyl)-5-(3,4-dimethoxyphenyl)-4′,5′-diphenyl-1,2′-biimidazole.

Hexaarylbiimidazole compounds have been used in the prior art according to US 2017/0266081 A1 in combination with co-initiators which are thiol group-containing heterocyclic aromatic compounds, in particular benzothiazole-and triazole-based compounds, namely with MMT (3-mercapto-4-methyl-4H-1,2,4-triazole) and MBT (2-mercaptobenzothiazole).

In the context of this invention, the inventors have found that the objectives described above are fulfilled advantageously if mercaptotetrazole compounds are used instead of the co-initiators known from the prior art. Corresponding mercaptotetrazole compounds are generally known from other areas of application and are commercially available from various manufacturers, e.g. from the company Merck.

During their own development work, the inventors found that advantageous initiator systems can be obtained in particular by using substituted mercaptotetrazole compounds. Therefore, a radiation-curing composition according to the invention is preferred, wherein the one or more mercaptotetrazole compounds are selected from the group consisting of mercaptotetrazole compounds having at least one substituent on the tetrazole ring, preferably exactly one substituent on the tetrazole ring, preferably selected from the group consisting of mercaptotetrazole compounds having exactly one substituent adjacent to the thiol group. A radiation-curing composition according to the invention is particularly preferred, wherein the substituent is selected from the group consisting of substituents with a +M effect, and/or wherein the substituent is selected from the group consisting of unsubstituted or substituted aryl groups, preferably substituted aryl groups with a substituent in para-position to the tetrazole ring, wherein the substituent is preferably selected from the group consisting of hydroxy groups and alkoxy groups, preferably from the group consisting of hydroxy groups and alkoxy groups with 1 to 3 carbon atoms, preferably hydroxy groups and alkoxy groups with 1 or 2 carbon atoms.

Among the mercaptotetrazole compounds in question, the inventors were able to identify examples which are particularly advantageous in combination with the hexaarylbiimidazole compounds to be used according to the invention, in particular in the specific low-filler radiation-curing compositions of this invention. In particular, a radiation-curing composition according to the invention is preferred, wherein the one or more mercaptotetrazole compounds are selected from the group consisting of 5-mercapto-1-phenyl-1H-tetrazole (MPHTA), 1-(4-hydroxyphenyl)-5-mercapto-1H-tetrazole (HPMTA), 1-(4-ethoxyphenyl)-5-mercapto-1H-tetrazole (EPMATA), 1-(4-carboxyphenyl)-5-mercapto-1H-tetrazole, 4-(5-sulfanyl-1H-1,2,3,4-tetrazol-1yl) benzonitrile (STABN) and 1-[4-(5-mercapto-1H-tetrazol-1-yl) phenyl] ethanone (MTPE). In addition or alternatively, a radiation-curing composition according to the invention is preferred, wherein the one or more mercaptotetrazole compounds are selected from the group consisting of 5-mercapto-1-phenyl-1H-tetrazoles (MPHTA), 1-(4-hydroxyphenyl)-5-mercapto-1H-tetrazole (HPMTA) and 1-(4-ethoxyphenyl)-5-mercapto-1H-tetrazole (EPMTA).

In accordance with the above description of the combined use of two or more different hexaarylbiimidazole compounds, the inventors propose that different mercaptotetrazole compounds can also be used, in particular in order to adapt the polymerization behavior precisely to the respective application requirements. A radiation-curing composition according to the invention is preferred, wherein the radiation-curing composition comprises two or more different mercaptotetrazole compounds.

In addition or alternatively, a radiation-curing composition according to the invention is also preferred, wherein the radiation-curing composition comprises as an hexaarylbiimidazole compound 2, 2′-bis (2-chlorophenyl)-4, 4′, 5, 5′-tetraphenyl-1, 2′-biimidazole and as an mercaptotetrazole compound 5-mercapto-1-phenyl-1H-tetrazole (MPHTA).

It can be seen as an advantage of the radiation-curing compositions according to the invention that they are very compatible with the presence of conventional additives, so that an advantageous property profile adapted to the respective application can be created by using such additives. In many cases, a radiation-curing composition according to the invention is preferred, wherein the radiation-curing composition comprises one or more additives, preferably with a combined mass fraction in the range from 0.01 to 10%, preferably in the range from 0.05 to 5%, particularly preferably in the range from 0.1 to 2%, wherein the additives are preferably selected from the group consisting of colorants, flow improvers, thixotropic agents, thickeners, stabilizers and UV-protective agents.

Radiation-curing compositions are disclosed below which, in the opinion of the inventors, are particularly preferred and which, in particularly preferred embodiments, are combined with one, two or more of the features described above as preferred.

In a first preferred embodiment, a radiation-curing composition is a radiation-curing composition according to the invention, comprising, based on the total mass of the radiation-curing composition:
(i) one or more radically polymerizable monomers in a combined mass fraction of 70% or more,
ii) one or more fillers in a combined mass fraction in the range from 0.05 to 29%,
iii) one or more hexaarylbiimidazole compounds in a combined mass fraction in the range from 0.1 to 5%, and
iiiv) one or more mercaptotetrazole compounds in a combined mass fraction in the range from 0.1 to 5%,
wherein the combined mass fraction of fillers in the radiation-curing composition is less than 30%.

In a second preferred embodiment, a radiation-curing composition is a radiation-curing composition according to the invention, comprising, based on the total mass of the radiation-curing composition:
i) one or more radically polymerizable monomers in a combined mass fraction of 70% or more, wherein the one or more radically polymerizable monomers are selected from the group consisting of (meth) acrylic acid, (meth) acrylates, (meth) acrylamides and other vinyl compounds,
ii) one or more fillers in a combined mass fraction in the range from 0.5 to 22.5%,
iii) one or more hexaarylbiimidazole compounds in a combined mass fraction in the range of 0.1 to 5%, wherein the one or more hexaarylbiimidazole compounds are selected from the group consisting of 2,2′-bis (2-chlorophenyl)-4, 4′,5,5′-tetraphenyl-1,2′-biimidazole 2,2′,4-tris (2-chlorophenyl)-5-(3,4-dimethoxyphenyl)-4′,5′-diphenyl-1,2′-biimidazole and 2,2′,4-tris (2-chlorophenyl)-5-(3,4- dimethoxyphenyl)-4′,5′-diphenyl-1,1′-biimidazole, and
iiiv) one or more mercaptotetrazole compounds in a combined mass fraction in the range of 0.1 to 5%, wherein the one or more mercaptotetrazole compounds are selected from the group consisting of 5-mercapto-1-phenyl-1H-tetrazoles (MPHTA), 1-(4-hydroxyphenyl)-5-mercapto-1H-tetrazole (HPMTA) and 1-(4-ethoxyphenyl)-5-mercapto-1H-tetrazole (EPMTA),
wherein the combined mass fraction of fillers in the radiation-curing composition is less than 25%.

In a third preferred embodiment, a radiation-curing composition is a radiation-curing composition according to the invention, comprising, based on the total mass of the radiation-curing composition:
i) one or more radically polymerizable monomers in a combined mass fraction of 75% or more, wherein the one or more radically polymerizable monomers are selected from the group consisting of monofunctional (meth) acrylates and polyfunctional (meth) acrylates,
ii) one or more fillers in a combined mass fraction in the range from 1 to 20%,
iii) one or more hexaarylbiimidazole compounds in a combined mass fraction in the range of 0.2 to 4%, wherein the one or more hexaarylbiimidazole compounds are selected from the group consisting of 2,2′-bis (2-chlorophenyl)-4, 4′,5,5′-tetraphenyl-1,2′-biimidazole 2,2′,4-tris (2-chlorophenyl)-5-(3,4-dimethoxyphenyl)-4′,5′-diphenyl-1,2′-biimidazole and 2,2′,4-tris (2-chlorophenyl)-5-(3,4-dimethoxyphenyl)-4′,5′-diphenyl-1,1′-biimidazole, and
iiiv) one or more mercaptotetrazole compounds in a combined mass fraction in the range of 0.2 to 4%, wherein the one or more mercaptotetrazole compounds are selected from the group consisting of 5-mercapto-1-phenyl-1H-tetrazoles (MPHTA), 1-(4-hydroxyphenyl)-5-mercapto-1H-tetrazole (HPMTA) and 1-(4-ethoxyphenyl)-5-mercapto-1H-tetrazole (EPMTA),
wherein the combined mass fraction of fillers in the radiation-curing composition is less than 25%,
wherein the radiation-curing composition has a dynamic viscosity in the range of 0.001 to 10 Pa s at 23° C.

The invention also relates to a dental component produced or producible using a DLP or SLA process by curing by radiation a radiation-curing composition according to the invention, in particular a preferred radiation-curing composition according to the invention.

In view of the above, it is preferred that a dental component according to the invention is produced or can be produced in a DLP process or SLA process by radiation curing of a radiation-curing composition with electromagnetic radiation in the wavelength range from 200 to 420 nm, preferably in the wavelength range from 250 to 415 nm, particularly preferably in the wavelength range from 300 to 410 nm.

The invention also relates to the use of an initiator system in a radiation-curing composition to improve the mechanical properties of dental components which can be produced from it using the DLP process or SLA process to reduce health concerns, wherein the radiation-curing composition comprises, based on the total mass of the radiation-curing composition:
(i) one or more radically polymerizable monomers in a combined mass fraction of 60% or more,
wherein the combined mass fraction of fillers in the radiation-curing composition is less than 30%,
wherein the initiator system comprises, based on the total mass of the radiation curing composition:
x) one or more hexaarylbiimidazole compounds in a combined mass fraction in the range from 0.1 to 5%, and
y) one or more mercaptotetrazole compounds in a combined mass fraction in the range from 0.1 to 5%.

In the following, the invention and preferred embodiments of the invention are explained and described in more detail with reference to experiments.

### A. Preparation of the Radiation-Curing Compositions:

The radiation-curing compositions were produced by mixing the components in the usual way.

The effect of the initiator systems was investigated on the basis of a model system M1, not according to the invention, which, in the experience of the inventors, is highly suitable as a starting point for a study on the effect of the initiator systems. The model system M1 had the composition shown in Table 1.

TABLE 1
Composition of model system M1

| Trade name | Chemical name | Mass fraction/% |
|---|---|---|
| Sartomer | Ethoxylated 2 bisphenol A | 4.50 |
| SR348L | dimethacrylate | |
| Sartomer | Tricyclodecane methanol | 28.40 |
| SR789 | acrylate | |
| Genomer | Urethane dimethacrylate | 46.00 |
| 4297 | | |
| Genomer | Carboxy-functionalized | 6.90 |
| 7151 | polyester acrylate | |
| Omnirad 819 | Phenylbis (2,4,6- | 1.00 |
| | trimethylbenzoyl) phosphine | |
| | oxide | |
| Aerosil ox. | Precipitated silicon dioxide | 9.00 |
| 50 sil. | | |
| Zirkonsil 520 | Mixture of zirconium and | 4.00 |
| | silicon dioxide | |
| Eusolex 4360 | Butylated hydroxytoluene | 0.20 |
| (UV09) | | |
| Lumilux Blue | Terephthalic acid ester | 0.006 |

Starting from the model system, radiation-curing compositions were prepared by replacing the phenylbis (2,4,6-trimethylbenzoyl) phosphine oxide with another initiator system. If a larger total mass fraction of the initiator system was set, the relative mass fraction of the other components was reduced accordingly. The amount of co-initiator (MTA, MPHTA, HPMTA and EPMTA) was calculated and added based on the amount of the added sensitizer (HABI), whereby the ratio of the amount of substance was partially varied.

The radiation-curing compositions C1 to C17 summarized in Table 2 were produced. In this case, the composition C1 is not a composition according to the invention.

TABLE 2
Composition of the radiation-curing compositions
C1 to C17 (all data in mass fractions)

| No. | HABI-1 | HABI-2 | HABI-3 | MTA | MPHTA | HPMTA | EPMTA |
|---|---|---|---|---|---|---|---|
| C1 | 1.0 | | | 0.46 | | | |
| C2 | 1.0 | | | | 0.81 | | |
| C3 | 1.0 | | | | | 0.88 | |
| C4 | 1.0 | | | | | | 1.01 |
| C5 | 0.5 | | | | | | 0.5 |
| C6 | 1.5 | | | | | | 1.52 |
| C7 | 2.0 | | | | 1.62 | | |
| C8 | 1.0 | | | | 0.27 | | |
| C9 | 1.0 | | | | 0.54 | | |
| C10 | 1.0 | | | | 1.35 | | |
| C11 | 1.0 | | | | 0.71 | | |
| C12 | 0.5 | 0.5 | | | 0.76 | | |
| C13 | 0.35 | 0.45 | | | 0.61 | | |
| C14 | 0.18 | 0.42 | | | 0.44 | | |
| C15 | | | 1.0 | | 0.71 | | |
| C16 | | | 0.5 | | 0.36 | | |
| C17 | 0.5 | | 0.5 | | 0.71 | | |

The abbreviations used above correspond to the compounds shown in Table 3.

TABLE 3
Compounds used

| Abbr. | Formula | Structure | Note |
|---|---|---|---|
| HABI-1 | 2,2′-Bis(2- chlorophenyl)- 4,4′,5,5′- tetraphenyl-1,2′- biimidazole | 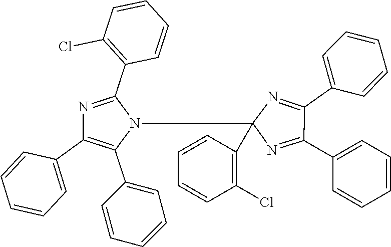 [SMILES: Clc1ccccc1-c1nc(-c2ccccc2)c(-c2ccccc2)n1C1(c2ccccc2Cl)N=C(c2ccccc2)C(c2ccccc2)=N1] | |
| HABI-2 | 2,2′,4-Tris(2- chlorophenyl)-5- (3,4- dimethoxyphenyl)- 4′,5′-diphenyl-1,2′- biimidazole | 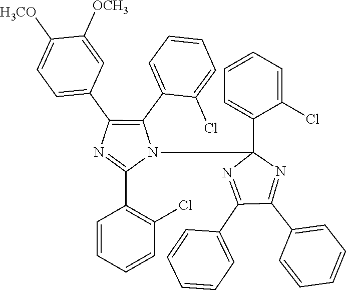 [SMILES: COc1ccc(-c2nc(-c3ccccc3Cl)n(C3(c4ccccc4Cl)N=C(c4ccccc4)C(c4ccccc4)=N3)c2-c2ccccc2Cl)cc1OC] | |
| HABI-3 | 2,2′,4-Tris(2- chlorophenyl)-5- (3,4- dimethoxyphenyl)- 4′,5′-diphenyl-1,1′- biimidazole | 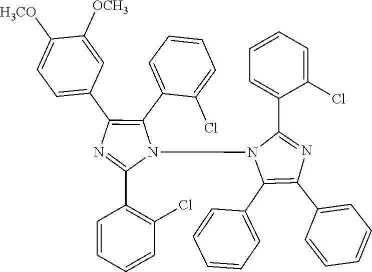 [SMILES: COc1ccc(-c2nc(-c3ccccc3Cl)n(-n3c(-c4ccccc4Cl)nc(-c4ccccc4)c3-c3ccccc3)c2-c2ccccc2Cl)cc1OC] | |
| MTA | 3-mercapto-1,2,4- triazole | 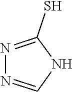 | |
| MPHTA | 5-mercapto-1- phenyl-1H- tetrazole | 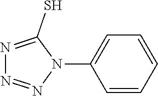 [SMILES: Sc1nnnn1-c1ccccc1] | +M effect weak |
| HPMTA | 1-(4- hydroxyphenyl)-5- mercapto-1H- tetrazole | 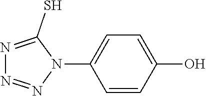 [SMILES: Oc1ccc(-n2nnnc2S)cc1] | +M effect pronounced |
| EPMTA | 1-(4-ethoxyphenyl)- 5-mercapto-1H- tetrazole | 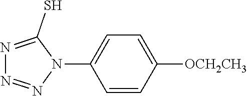 [SMILES: CCOc1ccc(-n2nnnc2S)cc1] | +M effect |

### B. Reactivity Experiments:

First, the reactivity and curing kinetics of the samples produced were investigated. For this purpose, the radiation-curing compositions were exposed to different doses of radiation and the resulting average layer thickness was determined.

The DLP printer (cara® Print 4.0 pro) was activated at least 5 minutes before use. The measurements were carried out at an ambient temperature between 21° C. and 25° C., where the temperature of the compositions being measured must not fall below 21° C. and must not exceed 25° C. The vat, i.e. the reservoir container for the photopolymer to be printed, into which an exposure window made of coated glass is inserted, was inserted into the printer and Hostaphan film (3 cm×6 cm) was placed in the center of the vat film. Initially, the exposure intensity was determined using a suitable radiometer (Opsytec Dr. Gröbel RM 12), whereby measurements were taken both with and without hostaphane film. A pea-sized drop of the composition was then applied to the Hostaphan film using a disposable plastic pipette and irradiated with a defined exposure dose. As soon as the exposure process was completed, the Hostaphan film with the partially cured material was removed from the vat and placed under a digital dial gauge (Sylvac S_Dial Work Nano) zeroed with regard to the Hostaphan film. After one minute, the measured value in the form of the resulting layer thickness was read and recorded. Four measurements were carried out for each exposure dose. The average layer thickness is calculated from the arithmetic mean of the determined layer thicknesses.

The results obtained are summarized in Table 4 below.

TABLE 4
Results of the reactivity experiments

| No. | Dose/(mJ/cm²) | In(dose) | Average layer thickness/μm |
|---|---|---|---|
| M1 | 5.570 | 1.7174 | 49.9 |
| | 7.798 | 2.0539 | 112.6 |
| | 10.026 | 2.3052 | 157.1 |
| | 12.254 | 2.5059 | 191.5 |
| | 15.596 | 2.7470 | 231.3 |
| C1 | 15.465 | 2.7386 | 41.2 |
| | 16.496 | 2.8031 | 100.0 |
| | 17.527 | 2.8637 | 159.1 |
| | 20.620 | 3.0263 | 265.6 |
| | 30.930 | 3.4317 | 502.1 |
| C2 | 16.496 | 2.8031 | 60.8 |
| | 17.527 | 2.8637 | 102.3 |
| | 20.620 | 3.0263 | 204.2 |
| | 23.713 | 3.1660 | 276.0 |
| | 30.930 | 3.4317 | 423.3 |
| C3 | 15.465 | 2.7386 | 72.3 |
| | 16.496 | 2.8031 | 123.6 |
| | 20.620 | 3.0263 | 262.7 |
| | 25.775 | 3.2494 | 386.7 |
| | 30.930 | 3.4317 | 475.6 |
| C4 | 16.496 | 2.8031 | 53.4 |
| | 17.527 | 2.8637 | 104.9 |
| | 21.651 | 3.0751 | 232.8 |
| | 25.775 | 3.2494 | 324.0 |
| | 30.930 | 3.4317 | 420.0 |
| C5 | 35.904 | 3.5808 | 51.5 |
| | 36.960 | 3.6098 | 91.6 |
| | 39.072 | 3.6654 | 157.2 |
| | 42.240 | 3.7434 | 241.0 |
| | 52.800 | 3.9665 | 409.0 |
| C6 | 12.372 | 2.5154 | 68.8 |
| | 13.403 | 2.5955 | 120.4 |
| | 14.434 | 2.6696 | 163.9 |
| | 16.496 | 2.8031 | 216.3 |
| | 20.620 | 3.0263 | 314.8 |
| C7 | 12.396 | 2.5174 | 87.3 |
| | 13.429 | 2.5974 | 116.8 |
| | 14.462 | 2.6715 | 145.7 |
| | 16.528 | 2.8051 | 188.9 |
| | 20.660 | 3.0282 | 269.1 |
| C8 | 21.500 | 3.0681 | 47.0 |
| | 22.575 | 3.1168 | 89.4 |
| | 24.725 | 3.2078 | 151.8 |
| | 27.950 | 3.3304 | 220.7 |
| | 32.250 | 3.4735 | 295.0 |
| C9 | 19.350 | 2.9627 | 61.1 |
| | 21.500 | 3.0681 | 136.0 |
| | 23.650 | 3.1634 | 192.3 |
| | 27.950 | 3.3304 | 280.8 |
| | 32.250 | 3.4735 | 356.2 |
| C10 | 16.125 | 2.7804 | 53.8 |
| | 17.200 | 2.8449 | 101.2 |
| | 19.350 | 2.9627 | 179.5 |
| | 23.650 | 3.1634 | 287.6 |
| | 26.875 | 3.2912 | 352.9 |
| C11 | 5.165 | 1.6419 | 69.7 |
| | 7.231 | 1.9784 | 95.4 |
| | 10.330 | 2.3351 | 124.4 |
| | 14.462 | 2.6715 | 152.5 |
| | 20.660 | 3.0282 | 187.2 |
| C12 | 5.285 | 1.6649 | 49.4 |
| | 7.399 | 2.0013 | 106.7 |
| | 9.513 | 2.2527 | 151.5 |
| | 13.741 | 2.6204 | 218.5 |
| | 21.140 | 3.0512 | 304.6 |
| C13 | 6.342 | 1.8472 | 60.2 |
| | 7.399 | 2.0013 | 90.6 |
| | 9.513 | 2.2527 | 140.7 |
| | 13.741 | 2.6204 | 212.1 |
| | 21.140 | 3.0512 | 305.9 |
| C14 | 7.399 | 2.0013 | 63.2 |
| | 9.513 | 2.2527 | 117.0 |
| | 11.627 | 2.4533 | 159.0 |
| | 14.798 | 2.6945 | 210.6 |
| | 21.140 | 3.0512 | 292.5 |
| C15 | 7.658 | 2.0358 | 54.1 |
| | 10.940 | 2.3924 | 64.6 |
| | 21.880 | 3.0856 | 101.6 |
| | 32.820 | 3.4910 | 115.5 |
| | 54.700 | 4.0019 | 164.9 |
| C16 | 25.979 | 2.9957 | 43.6 |
| | 38.969 | 3.4012 | 78.0 |
| | 51.959 | 3.6889 | 109.6 |
| | 64.948 | 3.9120 | 132.5 |
| | 103.918 | 4.3820 | 206.0 |
| C17 | 6.564 | 1.8816 | 43.8 |
| | 10.940 | 2.3924 | 74.9 |
| | 21.880 | 3.0856 | 116.8 |
| | 32.820 | 3.4910 | 146.5 |
| | 65.640 | 4.1842 | 212.3 |

From these measured values, the minimum exposure dose Emin (as the intersection of the linear regression with the x-axis) and the relationship between the generated layer thickness and light dose as dp (as the linear regression dose) can be derived from the linear regression by plotting layer thickness against In(dose). The results are summarized in Table 5.

TABLE 5
Minimum exposure dose Emin and regression slope dp

| No. | Eₘᵢₙ/(mJ/cm²) | dp |
|---|---|---|
| M1 | 1.42 | 250.80 |
| C1 | 14.07 | 649.70 |
| C2 | 14.66 | 572.38 |
| C3 | 13.39 | 580.41 |
| C4 | 14.73 | 575.68 |
| C5 | 33.27 | 911.35 |
| C6 | 10.43 | 468.95 |
| C7 | 9.65 | 353.61 |
| C8 | 19.54 | 602.93 |
| C9 | 17.10 | 568.92 |
| C10 | 14.48 | 580.30 |
| C11 | 2.31 | 84.33 |
| C12 | 4.12 | 183.94 |
| C13 | 4.75 | 203.11 |
| C14 | 5.57 | 217.70 |
| C15 | 3.18 | 54.308 |
| C16 | 14.82 | 116.28 |
| C17 | 3.86 | 71.781 |

It can be seen from the above data that excellent results can be achieved with radiation-curing compositions according to the invention, where in particular the curing kinetics can be specifically adapted over a wide range to the application requirements.

In particular, it is possible to achieve values for Eₘᵢₙ and dp that are comparable to those of the established phenylbis (2,4,6-trimethylbenzoyl) phosphine oxide. In particular, the combination of two hexaarylbiimidazole compounds in C12, C13 and C14 results in excellent values that are very close to the optimal values with regard to both parameters, which should be neither too low nor too high, for example to prevent premature, unwanted curing. By combining two hexaarylbiimidazole compounds, it is possible in particular to reduce the total content in samples C13 and C14 while maintaining advantageous curing properties, which is not only preferable from a manufacturing point of view, but also provides advantages with regard to the color properties of the compositions.

### C. Testing of Mechanical Properties:

In addition to the above experiments, the mechanical properties of selected radiation-curing compositions were investigated. For this purpose, the radiation-curing compositions were cured (DLP printer: cara® Print 4.0 pro; post-exposure: Twice five minutes with HiLite® power 3D) and the flexural strength, modulus of elasticity, work of fracture and fracture toughness were determined as follows:

The production of the flexible rods and the determining the flexural strength and the modulus of elasticity (E-modulus) are carried out in accordance with DIN EN ISO10477:2020 (7.5).

The test specimens are manufactured and the work of fracture and fracture toughness are determined in accordance with DIN EN ISO 20795-2:2013 (8.4).

The results are summarized in Table 6.

TABLE 6
Mechanical properties of the polymers

| | Flexural | | Work of | Fracture |
|---|---|---|---|---|
| | strength/ | E-modulus/ | fracture/ | toughness/ |
| No. | MPa | MPa | (J/m²) | (MPa m^{1/2}) |
| M1 | 110.4 | 2971 | 67.23 | 0.92 |
| C1 | 127.2 | 3289 | 63.18 | 0.90 |
| C2 | 129.4 | 3537 | 79.51 | 0.96 |
| C7 | 125.3 | 3528 | 80.45 | 0.99 |
| C12 | 128.0 | 3528 | 65.98 | 0.91 |

It can be seen from the above data that excellent mechanical properties can be achieved with radiation-curing compositions according to the invention, which impressively demonstrate for the directly comparable samples M1, C1 and C2 that the radiation-curing compositions according to the invention are clearly superior to the systems known from the prior art. The measurements on C7 and C12 confirm that the advantageous properties profiles can also be maintained over a wide composition range.

1. A radiation-curing composition for the manufacture of dental components using the digital light processing (DLP) process or stereolithography (SLA) process, comprising, based on the total mass of the radiation-curing composition:
(i) one or more radically polymerizable monomers in a combined mass fraction of 60% or more,
ii) one or more hexaarylbiimidazole compounds in a combined mass fraction in the range from 0.1 to 5%, and
iii) one or more mercaptotetrazole compounds in a combined mass fraction in the range from 0.1 to 5%,
wherein the combined mass fraction of fillers in the radiation-curing composition is less than 30%.

2. The radiation-curing composition according to claim 1, wherein the radiation-curing composition has a dynamic viscosity in the range of 0.001 to 10 Pas at 23° C.

3. The radiation-curing composition of claim 1, wherein the radiation-curing composition comprises one or more radically polymerizable monomers in a combined mass fraction of 65% or more.

4. The radiation-curing composition according to claim 1, wherein the combined mass fraction of fillers in the radiation-curing composition is 25% or less.

5. The radiation-curing composition according to claim 1, wherein the radiation-curing composition additionally comprises:
iv) one or more fillers in a combined mass fraction in the range from 0.05 to 29%.

6. The radiation-curing composition according to claim 1, wherein the quotient of the combined molar fraction of the one or more hexaarylbiimidazole compounds divided by the combined molar fraction of the one or more mercaptotetrazole compounds is 1.0 or less.

7. The radiation-curing composition according to claim 1, wherein the one or more hexaarylbiimidazole compounds are selected from the group consisting of hexaaryl-1,2′-biimidazole compounds.

8. The radiation-curing composition according to claim 1, wherein the radiation-curing composition comprises two or more different hexaarylbiimidazole compounds.

9. The radiation-curing composition according to claim 1, wherein the one or more mercaptotetrazole compounds are selected from the group consisting of mercaptotetrazole compounds having at least one substituent on the tetrazole ring.

10. The radiation-curing composition according to claim 9, wherein the substituent is selected from the group consisting of substituents having a +M effect.

11. The radiation-curing composition according to claim 9, wherein the one or more mercaptotetrazole compounds are selected from the group consisting of 5-mercapto-1-phenyl-1H-tetrazole (MPHTA), 1-(4-hydroxyphenyl)-5-mercapto-1H-tetrazole (HPMTA), 1-(4-ethoxyphenyl)-5-mercapto-1H-tetrazole (EPMATA), 1-(4-carboxyphenyl)-5-mercapto-1H-tetrazole, 4-(5-sulfanyl-1H-1,2,3,4-tetrazol-1yl) benzonitrile (STABN) and 1-[4-(5-mercapto-1H-tetrazol-1-yl) phenyl] ethanone (MTPE).

12. The radiation-curing composition according to claim 9, wherein the mercaptotetrazole compound is 1-(4-hydroxyphenyl)-5-mercapto-1H-tetrazole (HPMTA).

13. The radiation-curing composition according to claim 9, wherein the hexaarylbiimidazole compound is 2,2′-bis (2-chlorophenyl)-4, 4′, 5, 5′-tetraphenyl-1,2′-biimidazole and the mercaptotetrazole compound is 5-mercapto-1-phenyl-1H-tetrazole (MPHTA).

14. A dental component produced or producible in a DLP process or SLA process by radiation curing of a radiation-curing composition according to claim 1.

15. A method of manufacturing a dental component, the method comprising:
utilizing an initiator system in a radiation-curing composition, for improving the mechanical properties of dental components which can be produced from said radiation-curing composition using the DLP process or SLA process and for reducing health concerns, wherein the radiation-curing composition comprises, based on the total mass of the radiation-curing composition:
(i) one or more radically polymerizable monomers in a combined mass fraction of 60% or more,
wherein the combined mass fraction of fillers in the radiation-curing composition is less than 30%,
wherein the initiator system comprises, based on the total mass of the radiation curing composition:
x) one or more hexaarylbiimidazole compounds in a combined mass fraction in the range from 0.1 to 5%, and
y) one or more mercaptotetrazole compounds in a combined mass fraction in the range from 0.1 to 5%.
